(19) United States
(12) Patent Application Publication
(10) Pub. No.: US 2003/0051939 A1
(43) Pub. Date: Mar. 20, 2003

(54) Earpiece, for use on stethoscope, having a harder, load-bearing portion and a softer, acoustic sealing portion

(76) Inventor: Marc S. Werblud, Seattle, WA (US)

Correspondence Address: Robert W. Jenny, 319 - 101st SE, Apt. 313, Bellevue, WA, 98006, US

(21) Appl. No.: 09/961,007

(22) Filed: Sep. 20, 2001

(51) Int. Cl. A61B 7/02

(52) U.S. Cl. 181/131; 181/135

(57) ABSTRACT

The earpiece is characterized by having a harder portion which connects it to a stethoscope tube and supports its share of the weight of the stethoscope and a soft, compliant portion which extends farther into the ear passage than the hard portion and serves as the acoustic seal for the earpiece. In one embodiment the earpiece is a single piece made of foam plastic with the hardened portion including the surface of the lumen hardened using techniques well known in the art. In a second embodiment the harder portion is one piece and the softer, acoustic sealing portion a separate part which may be adhesively attached to the harder portion or be held in place by friction and therefore separately replaceable.

### BACKGROUND OF THE INVENTION

1. Field

The subject invention is in the field of equipment and devices which, in use, are inserted in people's ears, including earplugs, hearing aids or parts of hearing aids and stethoscope earpieces. In particular, the invention is in the field of earpieces of stethoscopes.

2. Prior Art

Much of the patented prior art in the general field is related to hearing aids and parts of hearing aids. U.S. Pat. Nos. 5,449,865 and 5,824,698 relate to earpieces for stethoscopes and the patents listed below relate to hearing aids.

| | |
|---|---|
| 5,002,151 | 5,798,743 |
| 5,654,530 | 5,129,174 |
| 5,742,692 | |

There are also commercially available earplugs, patented and not patented. All of the prior art pertains to one or more of the following features: (1) Conformity to ear passages for acoustic sealing; (2) detachable attachment of earpieces to stethoscope tubing or hearing aid assemblies; and (3) provision of replaceable coverings of earpieces for sanitation purposes.

Prior art compliant material earpieces or coverings for relatively hard ear pieces do not take into account the loads applied to the compliant materials by the weight of a stethoscope on which they are used. The weight is supported by the earpiece assemblies of the stethoscope. The result is that the compliant material is compacted between the harder portions of the earpieces and the lower portions of the ear passages into which the earpiece assemblies are inserted when in use. This compacting interferes with the primary function of the compliant materials, i.e. forming an effective acoustic seal between the earpiece and the inner surfaces of the ear passages.

Earpieces which have a relatively hard core portion and a conforming material outer portion have the disadvantage that the thickness of the conforming portion is limited, thereby limiting the amount of conformance provided.

In view of the prior art as described above, the primary objective of the subject invention is to provide a high conformance earpiece, for stethoscopes, the acoustic function of which is not compromised by the need to bear the weight of the stethoscope. A second objective is that the earpiece be economical enough to manufacture to be disposable for sanitation reasons.

### SUMMARY OF THE INVENTION

The subject invention is a load-bearing, highly conformable, disposable earpiece for a conventional stethoscope. The earpiece is made of elastomeric foam which, when deformed, has a relatively low restoring force so that, once it is deformed to closely fit an ear passage into which it is inserted, it exerts minimal pressure on the surface of the passage. The hole, or lumen, through the earpiece is kept from deforming by hardening or stiffening the surface layer of the lumen using one of a variety of known techniques available for the purpose. The selection of the technique depends in part on the specific material of which the earpiece is made and the techniques include (1) heating the material to be hardened; (2) impregnating the hole surface with self hardening liquid and (3) molding the wall of the lumen and then over molding the conformable elastomeric portion. Economics will also influence selection of the technique.

The provision of load-bearing capability which does not compromise acoustic sealing is achieved by making the portion of the earpiece which surrounds the end of the tube to which it is attached hard enough so that it does not compress appreciably when under load. This harder portion is the load-bearing portion of the earpiece and does not necessarily provide any acoustic sealing. The remainder of the earpiece extends further into the ear passage and provides the acoustic sealing. The earpiece may comprise a molded load-bearing portion with the acoustic sealing portion molded over it. Also, the two portions could be made separately and assembled to form the earpiece. In this case the acoustic sealing portion could be detachably attached to the load-bearing portion, using the hardened inner surface to retain a shape needed for the attachment, if necessary.

The invention is described in more detail below with reference to the attached drawings.

### BRIEF DESCRIPTION OF THE DRAWINGS

FIG. 1 illustrates one embodiment of the subject invention used on the ear end of a metal tube of a stethoscope.

FIG. 2 is a section taken at 2-2 in FIG. 1.

FIG. 3 is a section similar to that of FIG. 2 but showing an alternate embodiment.

FIG. 3A illustrates one embodiment of a flexible tubular portion.

FIG. 3B illustrates a second embodiment of a flexible tubular portion.

### DETAILED DESCRIPTION OF THE INVENTION

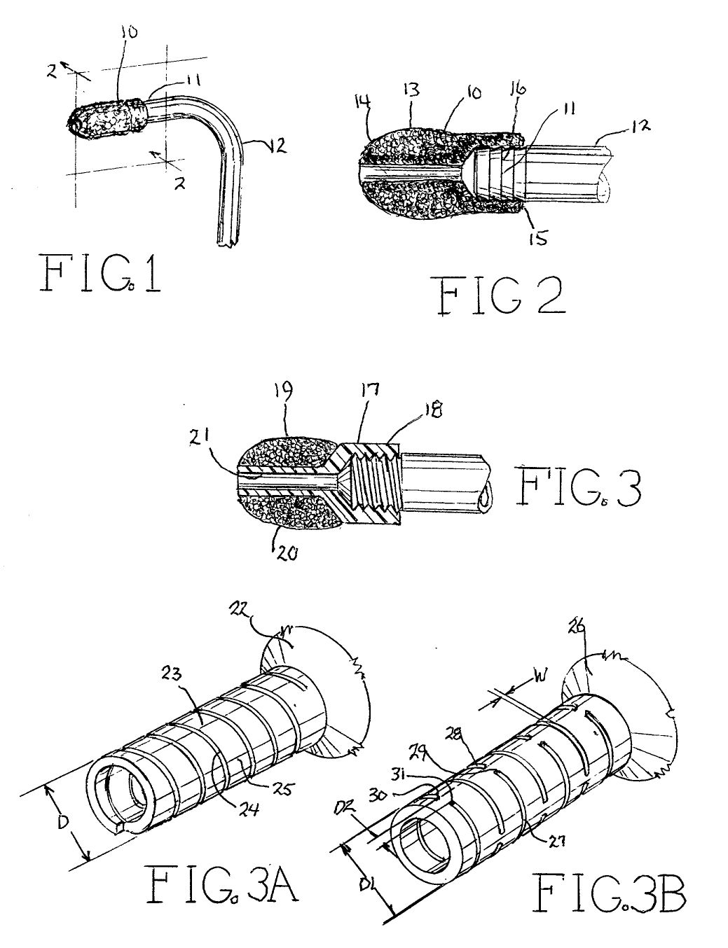

The subject invention is a load-bearing, highly conformable disposable earpiece for a stethoscope. FIG. 1 illustrates one embodiment of earpiece 10 installed on end 11 of stethoscope ear tube 12.

FIG. 2 is a section taken at 2-2 n FIG. 1. Earpiece 10 is made of elastomeric foam, preferably urethane because molded urethane foam parts inherently have a smooth outer shape and surface. At least some of the cells of foam are open so that the earpiece can be compressed to smaller diameters without changing significantly in length. The outer skin 13 is inherently porous enough to let the open cells breathe through it. The material is selected to have relatively small restoring forces. That is, it compresses easily when inserted into an ear canal and does not apply significant pressure on the canal surface as the material expands to form an acoustic seal. The surfaces of lumen 14 and counterbore 15 are hardened to a depth of several cells so that the lumen retains its shape when the piece is deformed on installation in an ear passage and so that the counterbore effectively engages the ridges, ridge 16 being typical, on end 11. Ridges of the configuration shown are commonly used on commercial fittings used with tubes and hoses in pneumatic equipment. However, the ridges may also be screw threads. The hardening can be done in any of several ways, including saturating the material portion to be hardened with hot glue which hardens when cooled. The glue is applied through a porous metal die which fits closely into the surfaces of the lumen and counterbore.

FIG. 3 is a section similar to that of FIG. 2 but showing an alternate embodiment of the subject invention. Earpiece 17 comprises two parts, a hard portion 18 and a compliant portion 19. The hard portion carries at least half of the weight of the stethoscope without significant deformation and the compliant portion provides acoustic sealing. The two parts may be adhesively attached or the compliant portion held in place by friction and therefore easily replaceable. The compliant portion is made of open cell elastomeric foam having a thin porous skin 20. Such material readily conforms to the shape and size of the ear passage into which it is inserted. Tubular portion 21 of the hard portion may be made flexible to more readily accommodate curvature of an ear passage.

FIG. 3A shows one embodiment of the tubular portion 22 made as a spiral strip 23 which can flex but still maintain the full diameter D of the tube. The gap 24 between turns, turn 25 being typical, is in a range of zero to 1/10 of width W of the strip. FIG. 3B illustrates a second embodiment for a flexible tubular portion 26. Pairs of equal and opposite slits, pair 27, 28 being typical, are oriented normal to the longitudinal axis of the earpieces with alternate pairs radially orthogonal to each other. In a preferred embodiment the width W of the slits is in a range of 1/10 to ¼ of the outside diameter D1 of the tubular portion and the distance d between slits is in a range of 1 to 10 times the width of the slits. The ends of the slit, end 29 being typical, are semi-cylindrical. The ends, ends 30 and 31 being typical, of each pair of slits are a distance D2 apart, the distance being in a range of 1/10 to ⅓ of the diameter of the tubular portion.

It is considered to be understandable from this description that the subject invention meets its objectives. It provides a high conformance earpiece for stethoscopes, the acoustic function of which is not compromised by the need for the earpieces to bear the weight of the stethoscope. Also, the earpiece is economical enough to manufacture to be disposable for sanitation reasons.

It is also considered to be understood other embodiments of the invention and modifications of the embodiments shown are possible within the scope of the invention which is limited only by the attached claims.

I claim:

1. An earpiece, for use on a stethoscope and being insertable in an ear passage, said stethoscope having a weight, said earpieces having an outer shape, a longitudinal centerline and a lumen having a surface and surrounding said longitudinal centerline, said earpiece comprising a harder portion whereby said earpiece can support at least half of said weight without significant deformation of said outer shape and said surface of said lumen, said earpiece further comprising a softer, compliant portion whereby said softer, compliant portion provides an acoustic seal at said outer surface and said ear passage.

2. The earpiece of claim 1 being made of two pieces, the first the harder portion, the second the compliant portion, said two pieces being attached adhesively.

3. The earpiece of claim 2 in which said two parts are held assembled by friction whereby said compliant part is removable and replaceable.

4. The earpiece of claim 2 in which said harder piece has a tubular portion forming said lumen and said tubular portion is made flexible by one or more slits in said tubular portion.

5. The earpiece of claim 3 in which said harder piece has a tubular portion forming said lumen and said tubular portion is made flexible by one or more slits in said tubular portion.
